(19) United States
(12) Patent Application Publication
(10) Pub. No.: US 2005/0276807 A1
(43) Pub. Date: Dec. 15, 2005

(54) Treatment of acne

(75) Inventors: Boris Skurkovich, Pawtucket, RI (US); Simon Skurkovich, Rockville, MD (US)

Correspondence Address: MORGAN, LEWIS & BOCKIUS LLP, 1701 MARKET STREET, PHILADELPHIA, PA, 19103-2921, US

(73) Assignee: Advanced Biotherapy, Inc.

(21) Appl. No.: 10/868,262

(22) Filed: Jun. 15, 2004

(51) Int. Cl. A61K 39/395

(52) U.S. Cl. 424/145.1

(57) ABSTRACT

The present invention includes methods for the treatment of acne comprising the administration of inhibitors of gamma interferon, tumor necrosis alpha and interleukin-1, and the administration of DMSO, retinoids and antibiotics, alone or in combination.

### BACKGROUND OF THE INVENTION

The ability of the mammalian immune system to recognize “self” versus “non-self” antigens is vital to successful host defense against invading microorganisms. “Self” antigens are those which are not detectably different from an animal's own constituents, whereas “non-self” antigens are those which are detectably different from or foreign to the mammal's constituents. A normal mammalian immune system functions to recognize “non-self antigens” and attack and destroy them. An autoimmune disorder such as for example, rheumatoid arthritis, insulin-independent diabetes mellitus, acquired immune deficiency syndrome (AIDS), multiple sclerosis, and the like, results when the immune system identifies “self” antigens as “non-self”, thereby initiating an immune response against the mammal's own body components (i.e., organs and/or tissues). This creates damage to the mammal's organs and/or tissues and can result in serious illness or death.

Predisposition of a mammal to an autoimmune disease is largely genetic; however, exogenous factors such as viruses, bacteria, or chemical agents may also play a role. Autoimmunity can also surface in tissues that are not normally exposed to lymphocytes such as for example, neural tissue. When a tissue not normally exposed to lymphocytes becomes exposed to these cells, the lymphocytes may recognize the surface antigens of these tissues as “non-self” and an immune response may ensue. Autoimmunity may also develop as a result of the introduction into the animal of antigens which are sensitive to the host's self antigens. An antigen which is similar to or cross-reactive with an antigen in an mammal's own tissue may cause lymphocytes to recognize and destroy both “self” and “non-self” antigens.

It has been suggested that the pathogenesis of autoimmune diseases is associated with a disruption in synthesis of interferons and other cytokines often induced by interferons (Skurkovich et al., Nature 217:551-552, 1974; Skurkovich et al., Annals of Allergy, 35:356, 1975; Skurkovich et al., J. Interferon Res. 12, Suppl. 1:S110, 1992; Skurkovich et al., Med. Hypoth., 41:177-185, 1993; Skurkovich et al., Med. Hypoth., 42:27-35, 1994; Gringeri et al., Cell. Mol. Biol. 41(3):381-387, 1995; Gringeri et al., J. Acquir. Immun. Defic. Syndr., 13:55-67, 1996). Cytokines are substances produced in different cell territories, including immune and nerve cells, which communicate with and affect the action of cells. In particular, interferon (IFN) gamma plays a significant pathogenic role in autoimmune dysfunction. gamma interferon stimulates cells to produce elevated levels of HLA class II antigens (Feldman et al., 1987, “Interferons and Autoimmunity”, In: IFN γ, p. 75, Academic Press). It is known that gamma interferon participates in the production of tumor necrosis factor (TNF), and it is also known that TNF also plays a role in stimulation of production of autoantibodies. In view of this, therapies to modulate these cytokines have been developed. Clinical success in treating several autoimmune diseases using antibodies to gamma interferon has been reported (Skurkovich et al., U.S. Pat. No. 5,888,511).

However, while an autoimmune response is considered to be typical in diseases such as multiple sclerosis and rheumatoid arthritis, one area of medicine where treatment of autoimmune or hyperimmune responses has not been fully explored is the area of skin diseases, particularly inflammatory skin diseases or skin diseases with an autoimmune component. Inflammation and autoimmune responses arising from antigens and the reaction of the skin to antigens is typical in skin diseases. Inflammation is the organism's normal reaction to invading foreign antigens.

Inflammation and autoimmune reactions in the skin are of considerable concern. Skin diseases including psoriasis, dermatitis, allergic conditions such as eczema, skin hypersensitivity reactions (including poison ivy and poison oak), decubitus ulcers, pressure ulcers, diabetic ulcers, epidermolysis bullosa, and milia psoriasis, atopic dermatitis, contact dermatitis, eczematoid dermatitis, seborrheic dermatitis, lichen planus, pemphigus, bullous pemphigoid, epidermolysis bullosa, urticaria, angioedema, vasculitides, erythema, dermal eosinophilia, acne, vitiligo and alopecia areata may also be the result of an inflammatory or autoimmune reaction in the skin.

Of these and other skin diseases, the most prevalent is acne. Acne, specifically acne vulgaris is a skin disease that is estimated to affect 85-100% of the population at one time in their life. Acne vulgaris is characterized by non-inflammatory follicular papules or comedones and by inflammatory papules, pustules and nodules. Areas of the skin with the most dense concentration of sebaceous follicles, usually the face, the upper chest and the back, are most commonly prone to acne vulgaris. Acne vulgaris is more common in men than in women during adolescence, but is more common in women during adulthood. Acne vulgaris may also occur in newborns, but often resolves when androgen levels begin to rise.

At least four factors are important in the development of acne lesions; follicular epidermal hyperproliferation and hyperkeratinization, excess sebum, Propionibacterium acnes, and inflammation. Follicular epidermal hyperproliferation and hyperkeratinization may be stimulated by increased levels of androgens and the alteration in the sebum and lipid levels in acne lesions. In addition, the presence of interleukin-1-alpha (IL-1α) may lead to hyperkeratinization and hyperproliferation of the infudibulum (Zouboulis, 2001, Dermatology 203: 277-279).

Excess sebum is strongly correlated with the degree and severity of acne lesions. Androgens stimulate sebum production and estrogens inhibit sebum production, and therefore an excess level of androgens or a hyperresponse to androgens may lead to acne lesion formation.

Propionibacterium acnes is a microaerophilic bacteria present in many acne lesions, but may not be present in the nascent acne lesion. It is widely accepted that the inflammation in acne is due to the immunological reaction to the extracellular products of P. acnes, such as free fatty acids (Zouboulis, 2001, Dermatology 203: 277-279). P. acnes may also bind to the toll-like receptors on monocytes, initiating the production of cytokines such as tumor necrosis factor, IL-12 and IL-8.

The inflammation of acne is likely the result of the immune response to P. acnes, as well as the inflammation of inflammatory cytokines. Such cytokines include bioactive IL-1-alpha, immunochemically detected IL-1-beta, and TNF-alpha (Ingham et al., 1992, J. Invest. Dermatol. 98: 895-901).

Acne vulgaris is characterized by comedones, papules, pustules, nodules and cysts distributed wherever sebaceous glands are present. In comedonal acne, no inflammatory lesions are present, mild inflammation usually involves papules and comedones, moderate inflammation involves comedones, inflammatory pustules and inflammatory pustules, and nodulocystic acne vulgaris is characterized by comedones, inflammatory lesions and large nodules greater than five millimeters in diameter. Acne fulminans is a severe inflammatory variant of acne that often presents as a systemic disease with symptoms including fever, arthritis and prominent acne vulgaris on the torso. Acne fulminans is often related to steroid use.

Acne often involves physical pain and can lead to scarring on prominent areas of the body, such as the face. More importantly, acne can lead to embarrassment and psychosocial suffering in afflicted individuals, especially in susceptible adolescents, even if acne is mild.

Treatment of acne usually comprises topical and systemic therapeutics. Salicylic acid washes help to ride comedones of excess sebum, and are generally a preventative measure. Benzoyl peroxide dries and peels the skin, prevents the growth of bacteria, and helps to clear blocked hair follicles. Over the counter formulations are readily available, and if necessary, stronger preparation can be prescribed. Tretinoin is a derivative of vitamin A that stimulates the turn over of skin cells, clearing the skin of plugged follicles. Tretinoin and retinoid analogs are also thought to inhibit the activity of leukocytes, pro-inflammatory cytokines and modulate the activity of transcription factors and toll receptors (Wolf, 2002, Adv. Ther. 19: 109-118). However, tretinoin also increases skin sensitivity to light, and therefore, if not used with sunscreen, can actually lead to increased skin damage. Topical antibiotics, such as clindamycin and erythromycin can be applied to affected areas to kill P. acnes and systemic antibiotics, often tetracycline or isotretinoin, can be prescribed to manage severe cases of acne. However, tetracycline increases the sensitivity of the skin to sunlight and can permanently stain the teeth of younger patients. Isotretinoin causes birth defects, and therefore sexually active women taking isotretinoin must use contraceptives to make absolutely sure that they do not become pregnant during or after isotretinoin treatment.

To date, there are few successful or long-term methods or compositions for effectively treating inflammatory and/or autoimmune reactions in the mammalian skin. The present invention provides such methods and compositions.

### SUMMARY OF THE INVENTION

The present invention includes a method of treating acne in a patient, the method comprising administering to the patient an effective amount of an antibody to gamma interferon, an antibody to tumor necrosis factor alpha, and an antibody to interleukin-1.

In one aspect of the present invention, the antibody is selected from the group consisting of a polyclonal antibody, a monoclonal antibody, a humanized antibody, a synthetic antibody, a heavy chain antibody, a biologically active fragment of an antibody, wherein the biologically active fragment is a Fab fragment, a F(ab′)₂ fragment, a Fv fragment, and combinations thereof.

In yet another aspect of the present invention, the antibody is administered by the route selected from the group consisting of intramuscularly, intravenously, intradermally, cutaneously, ionophoretically, topically, locally, and inhalation.

In still another aspect of the present invention, the antibody is administered topically.

In still another aspect of the present invention, the antibody is selected from the group consisting of a polyclonal antibody, a monoclonal antibody, a synthetic antibody, a heavy chain antibody, a humanized antibody, a biologically active fragment of an antibody, wherein the biologically active fragment is a Fab fragment, a F(ab′)₂ fragment, a Fv fragment, and combinations thereof.

In still another aspect of the present invention, the heavy chain antibody is selected from the group consisting of a camelid antibody, a heavy chain disease antibody, and a variable heavy chain immunoglobulin.

In one aspect of the invention, the method further comprises administering an antibiotic.

In yet another aspect of the invention, the method further comprises administering a retinoid.

The present invention also includes a method of treating acne in a patient, the method comprising administering to the patient an effective amount of an antibody to tumor necrosis factor alpha, and an antibody to interleukin-1.

In one aspect of the present invention, the antibody is selected from the group consisting of a polyclonal antibody, a monoclonal antibody, a humanized antibody, a synthetic antibody, a heavy chain antibody, a biologically active fragment of an antibody, wherein the biologically active fragment is a Fab fragment, a F(ab′)₂ fragment, a Fv fragment, and combinations thereof.

In yet another aspect of the present invention, the antibody is administered by the route selected from the group consisting of intramuscularly, intravenously, intradermally, cutaneously, ionophoretically, topically, locally, and inhalation.

In still another aspect of the present invention, the antibody is administered topically.

In still another aspect of the present invention, the antibody is selected from the group consisting of a polyclonal antibody, a monoclonal antibody, a synthetic antibody, a heavy chain antibody, a humanized antibody, a biologically active fragment of an antibody, wherein the biologically active fragment is a Fab fragment, a F(ab′)₂ fragment, a Fv fragment, and combinations thereof.

In yet another aspect of the present invention, the heavy chain antibody is selected from the group consisting of a camelid antibody, a heavy chain disease antibody, and a variable heavy chain immunoglobulin.

In one aspect of the invention, the method further comprises administering an antibiotic.

In yet another aspect of the invention, the method further comprises administering a retinoid.

The present invention also includes a method of treating acne in a patient, the method comprising administering to the patient an effective amount of an antibody to gamma interferon and an antibody to interleukin-1.

In one aspect of the present invention, the antibody is selected from the group consisting of a polyclonal antibody, a monoclonal antibody, a humanized antibody, a synthetic antibody, a heavy chain antibody, a biologically active fragment of an antibody, wherein the biologically active fragment is a Fab fragment, a F(ab′)₂ fragment, a Fv fragment, and combinations thereof.

In yet another aspect of the present invention, the antibody is administered by the route selected from the group consisting of intramuscularly, intravenously, intradermally, cutaneously, ionophoretically, topically, locally, and inhalation.

In still another aspect of the present invention, the antibody is administered topically.

In another aspect of the present invention, the antibody is selected from the group consisting of a polyclonal antibody, a monoclonal antibody, a synthetic antibody, a heavy chain antibody, a humanized antibody, a biologically active fragment of an antibody, wherein the biologically active fragment is a Fab fragment, a F(ab′)₂ fragment, a Fv fragment, and combinations thereof.

In yet another aspect of the present invention, the heavy chain antibody is selected from the group consisting of a camelid antibody, a heavy chain disease antibody, and a variable heavy chain immunoglobulin.

In one aspect of the invention, the method further comprises administering an antibiotic.

In yet another aspect of the invention, the method further comprises administering a retinoid.

The present invention also includes a method of treating acne in a patient, the method comprising administering to the patient an effective amount of an antibody to gamma interferon and an antibody to tumor necrosis factor alpha.

In one aspect of the present invention, the antibody is selected from the group consisting of a polyclonal antibody, a monoclonal antibody, a humanized antibody, a synthetic antibody, a heavy chain antibody, a biologically active fragment of an antibody, wherein the biologically active fragment is a Fab fragment, a F(ab′)₂ fragment, a Fv fragment, and combinations thereof.

In yet another aspect of the present invention, the antibody is administered by the route selected from the group consisting of intramuscularly, intravenously, intradermally, cutaneously, ionophoretically, topically, locally, and inhalation.

In still another aspect of the present invention, the antibody is administered topically.

In another aspect of the present invention, the antibody is selected from the group consisting of a polyclonal antibody, a monoclonal antibody, a synthetic antibody, a heavy chain antibody, a humanized antibody, a biologically active fragment of an antibody, wherein the biologically active fragment is a Fab fragment, a F(ab′)₂ fragment, a Fv fragment, and combinations thereof.

In yet another aspect of the present invention, the heavy chain antibody is selected from the group consisting of a camelid antibody, a heavy chain disease antibody, and a variable heavy chain immunoglobulin.

In one aspect of the invention, the method further comprises administering an antibiotic.

In yet another aspect of the invention, the method further comprises administering a retinoid.

The present invention also includes a method of treating acne in a patient, the method comprising administering to the patient an effective amount of an antibody to gamma interferon.

In one aspect of the present invention, the antibody is selected from the group consisting of a polyclonal antibody, a monoclonal antibody, a humanized antibody, a synthetic antibody, a heavy chain antibody, a biologically active fragment of an antibody, wherein the biologically active fragment is a Fab fragment, a F(ab′)₂ fragment, a Fv fragment, and combinations thereof.

In yet another aspect of the present invention, the antibody is administered by the route selected from the group consisting of intramuscularly, intravenously, intradermally, cutaneously, ionophoretically, topically, locally, and inhalation.

In still another aspect of the present invention, the antibody is administered topically.

In another aspect of the present invention, the antibody is selected from the group consisting of a polyclonal antibody, a monoclonal antibody, a synthetic antibody, a heavy chain antibody, a humanized antibody, a biologically active fragment of an antibody, wherein the biologically active fragment is a Fab fragment, a F(ab′)₂ fragment, a Fv fragment, and combinations thereof.

In yet another aspect of the present invention, the heavy chain antibody is selected from the group consisting of a camelid antibody, a heavy chain disease antibody, and a variable heavy chain immunoglobulin.

In one aspect of the invention, the method further comprises administering an antibiotic.

In yet another aspect of the invention, the method further comprises administering a retinoid.

The present invention also includes a method of treating acne in a patient, the method comprising administering to the patient an effective amount of an antibody to interleukin-1.

In one aspect of the present invention, the antibody is selected from the group consisting of a polyclonal antibody, a monoclonal antibody, a humanized antibody, a synthetic antibody, a heavy chain antibody, a biologically active fragment of an antibody, wherein the biologically active fragment is a Fab fragment, a F(ab′)₂ fragment, a Fv fragment, and combinations thereof.

In yet another aspect of the present invention, the antibody is administered by the route selected from the group consisting of intramuscularly, intravenously, intradermally, cutaneously, ionophoretically, topically, locally, and inhalation.

In still another aspect of the present invention, the antibody is administered topically.

In yet another aspect of the present invention, the antibody is selected from the group consisting of a polyclonal antibody, a monoclonal antibody, a synthetic antibody, a heavy chain antibody, a humanized antibody, a biologically active fragment of an antibody, wherein the biologically active fragment is a Fab fragment, a F(ab′)₂ fragment, a Fv fragment, and combinations thereof.

In still another aspect of the present invention, the heavy chain antibody is selected from the group consisting of a camelid antibody, a heavy chain disease antibody, and a variable heavy chain immunoglobulin.

In one aspect of the invention, the method further comprises administering an antibiotic.

In yet another aspect of the invention, the method further comprises administering a retinoid.

The present invention also includes a method of treating acne in a patient, the method comprising administering to the patient an effective amount of an antibody to tumor necrosis factor alpha.

In one aspect of the present invention, the antibody is selected from the group consisting of a polyclonal antibody, a monoclonal antibody, a humanized antibody, a synthetic antibody, a heavy chain antibody, a biologically active fragment of an antibody, wherein the biologically active fragment is a Fab fragment, a F(ab′)₂ fragment, a Fv fragment, and combinations thereof.

In yet another aspect of the present invention, the antibody is administered by the route selected from the group consisting of intramuscularly, intravenously, intradermally, cutaneously, ionophoretically, topically, locally, and inhalation.

In still another aspect of the present invention, the antibody is administered topically.

In another aspect of the present invention, the antibody is selected from the group consisting of a polyclonal antibody, a monoclonal antibody, a synthetic antibody, a heavy chain antibody, a humanized antibody, a biologically active fragment of an antibody, wherein the biologically active fragment is a Fab fragment, a F(ab′)₂ fragment, a Fv fragment, and combinations thereof.

In yet another aspect of the present invention, the heavy chain antibody is selected from the group consisting of a camelid antibody, a heavy chain disease antibody, and a variable heavy chain immunoglobulin.

In one aspect of the invention, the method further comprises administering an antibiotic.

In yet another aspect of the invention, the method further comprises administering a retinoid.

The present invention also includes a kit for treating acne in a patient, said kit comprising an antibody to gamma interferon, an antibody to tumor necrosis factor alpha, an antibody to interleukin-1, and a pharmaceutically acceptable carrier, said kit further comprising an applicator, and an instructional material for the use thereof.

The present invention also includes a kit for treating acne in a patient, said kit comprising an antibody to tumor necrosis factor alpha, an antibody to interleukin-1, and a pharmaceutically acceptable carrier, said kit further comprising an applicator, and an instructional material for the use thereof.

The present invention also includes a kit for treating acne in a patient, said kit comprising an antibody to gamma interferon, an antibody to interleukin-1, and a pharmaceutically acceptable carrier, said kit further comprising an applicator, and an instructional material for the use thereof.

The present invention also includes a kit for treating acne in a patient, said kit comprising an antibody to gamma interferon, an antibody to tumor necrosis factor alpha, and a pharmaceutically acceptable carrier, said kit further comprising an applicator, and an instructional material for the use thereof.

The present invention also includes a kit for treating acne in a patient, said kit comprising an antibody to gamma interferon and a pharmaceutically acceptable carrier, said kit further comprising an applicator, and an instructional material for the use thereof.

The present invention also includes a kit for treating acne in a patient, said kit comprising an antibody to interleukin-1 and a pharmaceutically acceptable carrier, said kit further comprising an applicator, and an instructional material for the use thereof.

The present invention also includes a kit for treating acne in a patient, said kit comprising an antibody to tumor necrosis factor alpha, and a pharmaceutically acceptable carrier, said kit further comprising an applicator, and an instructional material for the use thereof.

### DETAILED DESCRIPTION OF THE INVENTION

The present invention includes the discovery that administration of antibodies to gamma interferon, antibodies to tumor necrosis factor alpha (TNF-alpha), and antibodies to interleukin-1 (IL-1), alone or in combination, to a patient with an autoimmune disease or inflammation of the skin is in alleviating or eliminating the autoimmune reaction or inflammatory reaction. Such autoimmune and inflammatory skin diseases include acne, psoriasis, dermatitis, allergic conditions such as eczema, skin hypersensitivity reactions (including poison ivy and poison oak), decubitus ulcers, pressure ulcers, diabetic ulcers, epidermolysis bullosa, and milia psoriasis, atopic dermatitis, contact dermatitis, eczematoid dermatitis, seborrheic dermatitis, lichen planus, pemphigus, bullous pemphigoid, epidermolysis bullosa, urticaria, angioedema, vasculitides, erythema, dermal eosinophilia, vitiligo and alopecia areata. Autoimmune and inflammatory skin reactions may also occur as a result of contracting an infectious disease or as a result of other medications.

As disclosed herein, antibodies to gamma interferon, antibodies to TNF-alpha and antibodies to IL-1, alone or in combination, are useful for the treatment of autoimmune or inflammatory skin diseases such as acne. However, the invention should not be construed as being limited solely to the examples provided herein, as other autoimmune and inflammatory diseases of the skin which are at present unknown, once known, may also be treatable using the methods of the invention.

The invention includes a method of treating a skin disease characterized by a hyperimmune response in the skin of a mammal. The method comprises administering to a patient with a skin disease characterized by a hyperimmune response or inflammatory response an antibody to gamma interferon, an antibody to TNF-alpha and an antibody to IL-1, alone or in combination. The antibody can be administered using techniques well known in the art and disclosed elsewhere herein, including parenteral administration, such as intramuscular, intravenous, intradermal, cutaneous, subcutaneous or local administration. In addition, an antibody can be administered ionophoretically, topically, and via inhalation. Preferably, the antibody or combination of antibodies is administered, alone or in combination, to the skin topically. The method can be used to treat an autoimmune or inflammatory skin disease in any mammal; however, preferably, the mammal is a human.

The antibodies to gamma interferon useful in the methods of the invention may be polyclonal antibodies, monoclonal antibodies, synthetic antibodies, such as a biologically active fragment of an antibody to gamma interferon, or they may be humanized monoclonal antibodies. Methods of making and using each of the types of antibodies useful in the methods of the invention are now described. In addition, human antibodies to gamma interferon, TNF-alpha or IL-1, obtained from human donors, may be employed in the invention.

When the antibody used in the methods of the invention is a polyclonal antibody (IgG), the antibody is generated by inoculating a suitable animal with gamma interferon, TNF-alpha, IL-1, or a fragment thereof. Antibodies produced in the inoculated animal which specifically bind gamma interferon, TNF-alpha or IL-1 are then isolated from fluid obtained from the animal. Antibodies may be generated in this manner in several non-human mammals such as, but not limited to goat, sheep, horse, camel, rabbit, and donkey. Methods for generating polyclonal antibodies are well known in the art and are described, for example in Harlow, et al. (1988, In: Antibodies, A Laboratory Manual, Cold Spring Harbor, N.Y.).

When the antibody used in the methods of the invention is a monoclonal antibody, the antibody is generated using any well known monoclonal antibody preparation procedures such as those described, for example, in Harlow et al. (supra) and in Tuszynski et al. (1988, Blood, 72:109-115). Generally, monoclonal antibodies directed against a desired antigen are generated from mice immunized with the antigen using standard procedures as referenced herein. Monoclonal antibodies directed against full length or peptide fragments of gamma interferon, TNF-alpha or IL-1 may be prepared using the techniques described in Harlow, et al. (supra).

When the antibody used in the methods of the invention is a biologically active antibody fragment or a synthetic antibody corresponding to antibody to gamma interferon, an antibody to TNF-alpha or an antibody to IL-1, the antibody is prepared as follows: a nucleic acid encoding the desired antibody or fragment thereof is cloned into a suitable vector. The vector is transfected into cells suitable for the generation of large quantities of the antibody or fragment thereof. DNA encoding the desired antibody is then expressed in the cell thereby producing the antibody. The nucleic acid encoding the desired peptide may be cloned and sequenced using technology which is available in the art, and described, for example, in Wright et al. (1992, Critical Rev. in Immunol. 12(3,4):125-168) and the references cited therein. Alternatively, quantities of the desired antibody or fragment thereof may also be synthesized using chemical synthesis technology. If the amino acid sequence of the antibody is known, the desired antibody can be chemically synthesized using methods known in the art.

The present invention also includes the use of humanized antibodies specifically reactive with gamma interferon epitopes. The present invention further includes the use of humanized antibodies specifically reactive with TNF-alpha or IL-1 epitopes. These antibodies are capable of neutralizing human gamma interferon, human TNF-alpha or human IL-1. The humanized antibodies of the invention have a human framework and have one or more complementarity determining regions (CDRs) from an antibody, typically a mouse antibody, specifically reactive with gamma interferon, IL-1 or TNF-alpha. Thus, the humanized gamma interferon antibodies of the present invention are useful in the treatment of autoimmune or inflammatory skin diseases, such as acne, and other such diseases which are characterized by an autoimmune or inflammatory reaction which includes overproduction of gamma interferon, TNF-alpha and/or IL-1.

When the antibody used in the invention is humanized, the antibody may be generated as described in Queen, et al. (U.S. Pat. No. 6,180,370), Wright et al., (supra) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as human gamma interferon, TNF-alpha or IL-1, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well known procedures. Preferably, the human constant region DNA sequences are isolated from immortalized B-cells as described in WO 87/02671. CDRs useful in producing the antibodies of the present invention may be similarly derived from DNA encoding monoclonal antibodies capable of binding to human IFN gamma, human TNF-alpha or human IL-1. Such humanized antibodies may be generated using well known methods in any convenient mammalian source capable of producing antibodies, including, but not limited to, mice, rats, camels, llamas, rabbits, or other vertebrates. Suitable cells for constant region and framework DNA sequences and host cells in which the antibodies are expressed and secreted, can be obtained from a number of sources such as the American Type Culture Collection, Manassas, Va.

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies.

Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The use of Old World and New World camelids for the production of antibodies is contemplated in the present invention, as are other methods for the production of camelid antibodies set forth herein.

The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species. As an example, the production of antibodies in mammals is detailed in such references as Harlow et al., (1989, Antibodies: A Laboratory Manual, Cold Spring Harbor, N.Y.). Camelid species for the production of antibodies and sundry other uses are available from various sources, including but not limited to, Camello Fataga S. L. (Gran Canaria, Canary Islands) for Old World camelids, and High Acres Llamas (Fredricksburg, Tex.) for New World camelids.

The isolation of camelid antibodies from the serum of a camelid species can be performed by many methods well known in the art, including but not limited to ammonium sulfate precipitation, antigen affinity purification, Protein A and Protein G purification, and the like. As an example, a camelid species may be immunized to a desired antigen, for example gamma interferon, IL-1, or a TNF-alpha peptide, or fragment thereof, using techniques well known in the art. The whole blood can them be drawn from the camelid and sera can be separated using standard techniques. The sera can then be absorbed onto a Protein G-Sepharose column (Pharmacia, Piscataway, N.J.) and washed with appropriate buffers, for example 20 mM phosphate buffer (pH 7.0). The camelid antibody can then be eluted using a variety of techniques well known in the art, for example 0.15 M NaCl, 0.58% acetic acid (pH 3.5). The efficiency of the elution and purification of the camelid antibody can be determined by various methods, including SDS-PAGE, Bradford Assays, and the like. The fraction that is not absorbed can be bound to a Protein A-Sepharose column (Pharmacia, Piscataway, N.J.) and eluted using, for example, 0.15 M NaCl, 0.58% acetic acid (pH 4.5). The skilled artisan will readily understand that the above methods for the isolation and purification of camelid antibodies are exemplary, and other methods for protein isolation are well known in the art and are encompassed in the present invention.

The present invention further contemplates the production of camelid antibodies expressed from nucleic acid. Such methods are well known in the art, and are detailed in, for example U.S. Pat. Nos. 5,800,988; 5,759,808; 5,840,526, and 6,015,695, which are incorporated herein by reference in their entirety. Briefly, cDNA can be synthesized from camelid spleen mRNA. Isolation of RNA can be performed using multiple methods and compositions, including TRIZOL (Gibco/BRL, La Jolla, Calif.) further, total RNA can be isolated from tissues using the guanidium isothiocyanate method detailed in, for example, Sambrook et al. (1989, Molecular Cloning, A Laboratory Manual, Cold Spring Harbor, N.Y.). Methods for purification of MRNA from total cellular or tissue RNA are well known in the art, and include, for example, oligo-T paramagnetic beads. cDNA synthesis can then be obtained from mRNA using MRNA template, an oligo dT primer and a reverse transcriptase enzyme, available commercially from a variety of sources, including Invitrogen (La Jolla, Calif.). Second strand cDNA can be obtained from mRNA using RNAse H and E. coli DNA polymerase I according to techniques well known in the art.

Identification of cDNA sequences of relevance can be performed by hybridization techniques well known by one of ordinary skill in the art, and include methods such as Southern blotting, RNA protection assays, and the like. Probes to identify variable heavy immunoglobulin chains (V_{HH}) are available commercially and are well known in the art, as detailed in, for example, Sastry et al., (1989, Proc. Nat'l. Acad. Sci. USA, 86:5728). Full-length clones can be produced from cDNA sequences using any techniques well known in the art and detailed in, for example, Sambrook et al. (1989, Molecular Cloning, A Laboratory Manual, Cold Spring Harbor, N.Y.).

The clones can be expressed in any type of expression vector known to the skilled artisan. Further, various expression systems can be used to express the V_{HH} peptides of the present invention, and include, but are not limited to eukaryotic and prokaryotic systems, including bacterial cells, mammalian cells, insect cells, yeast cells, and the like. Such methods for the expression of a protein are well known in the art and are detailed elsewhere herein.

The V_{HH} immunoglobulin proteins isolated from a camelid species or expressed from nucleic acids encoding such proteins can be used directly in the methods of the present invention, or can be further isolated and/or purified using methods disclosed elsewhere herein.

The present invention is not limited to V_{HH} proteins isolated from camelid species, but also includes V_{HH} proteins isolated from other sources such as animals with heavy chain disease (Seligmann et al., 1979, Immunological Rev. 48:145-167, incorporated herein by reference in its entirety). The present invention further comprises variable heavy chain immunoglobulins produced from mice and other mammals, as detailed in Ward et al. (1989, Nature 341:544-546, incorporated herein by reference in its entirety). Briefly, V_{H} genes were isolated from mouse splenic preparations and expressed in E. coli. The present invention encompasses the use of such heavy chain immunoglobulins in the treatment of various autoimmune disorders detailed herein.

As used herein, the term “heavy chain antibody” or “heavy chain antibodies” comprises immunoglobulin molecules derived from camelid species, either by immunization with an peptide and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

Once expressed, whole antibodies, dimers derived therefrom, individual light and heavy chains, or other forms of antibodies can be purified according to standard procedures known in the art. Such procedures include, but are not limited to, ammonium sulfate precipitation, the use of affinity columns, routine column chromatography, gel electrophoresis, and the like (see, generally, R. Scopes, “Protein Purification”, Springer-Verlag, N.Y. (1982)). Substantially pure antibodies of at least about 90% to 95% homogeneity are preferred, and antibodies having 98% to 99% or more homogeneity most preferred for pharmaceutical uses. Once purified, the antibodies may then be used therapeutically.

In addition to the antibodies discussed above, other “substantially homologous” modifications to native gamma interferon, TNF-alpha or IL-1 antibody sequences can be readily designed and manufactured using various recombinant DNA techniques well known to those skilled in the art. Moreover, a variety of different human framework regions may be used singly or in combination as a basis for humanizing antibodies directed at gamma interferon, TNF-alpha and IL-1. In general, modifications of genes may be readily accomplished using a variety of well-known techniques, such as site-directed mutagenesis (Gillman and Smith, Gene, 8, 81-97 (1979); Roberts et al., 1987, Nature, 328, 731-734).

Substantially homologous sequences to a gamma interferon antibody sequence are those which exhibit at least about 85% homology, usually at least about 90%, and preferably at least about 95% homology with a reference gamma interferon immunoglobulin protein. Further, substantially homologous sequences to a TNF-alpha antibody sequence are those which exhibit at least about 85% homology, usually at least about 90%, and preferably at least about 95% homology with a reference TNF-alpha immunoglobulin protein. In addition, substantially homologous sequences to an IL-1 antibody sequence are those which exhibit at least about 85% homology, usually at least about 90%, and preferably at least about 95% homology with a reference IL-1 immunoglobulin protein.

Alternatively, polypeptide fragments comprising only a portion of the primary antibody structure may be produced, which fragments possess one or more functions of gamma interferon, TNF-alpha or IL-1 antibody. These polypeptide fragments may be generated by proteolytic cleavage of intact antibodies using methods well known in the art, or they may be generated by inserting stop codons at the desired locations in vectors comprising the fragment using site-directed mutagenesis.

DNA encoding an antibody to gamma interferon, TNF-alpha or IL-1 are expressed in a host cell driven by a suitable promoter regulatory sequence which is operably linked to the DNA encoding the antibody. Typically, DNA encoding the antibody is cloned into a suitable expression vector such that the sequence encoding the antibody is operably linked to the promoter/regulatory sequence. Such expression vectors are typically replication competent in a host organism either as an episome or as an integral part of the host chromosomal DNA. Commonly, an expression vector will comprise DNA encoding a detectable marker protein, e.g., a gene encoding resistance to tetracycline or neomycin, to permit detection of cells transformed with the desired DNA sequences (U.S. Pat. No. 4,704,362).

Escherichia coli is an example of a prokaryotic host which is particularly useful for expression of DNA sequences encoding the antibodies of the present invention. Other microbial hosts suitable for use include but are not limited to, Bacillus subtilis, and other enterobacteriaceae, such as selected member of Salmonella, Serratia, and various Pseudomonas species. It is possible to generate expression vectors suitable for the desired host cell wherein the vectors will typically comprise an expression control sequence which is compatible with the host cell. A variety of promoter/regulatory sequences are useful for expression of genes in these cells, including but not limited to the lactose promoter system, a tryptophan (trp) promoter system, a beta-lactamase promoter system, or a promoter system derived from phage lambda. The promoter will typically control expression of the antibody whose DNA sequence is operably linked thereto, the promoter is optionally linked with an operator sequence and generally comprises RNA polymerase and ribosome binding site sequences and the like for initiating and completing transcription and translation of the desired antibody.

Yeast is an example of a eukaryotic host useful for cloning DNA sequences encoding the antibodies of the present invention. Saccharomyces is a preferred eukaryotic host. Promoter/regulatory sequences which drive expression of nucleic acids in eukaryotic cells include but are not limited to the 3-phosphoglycerate kinase promoter/regulatory sequence and promoter/regulatory sequences which drive expression of nucleic acid encoding other glycolytic enzymes.

In addition to microorganisms, mammalian tissue cell culture may also be used to express and produce the antibodies of the present invention (Winnacker, 1987, “From Genes to Clones,” VCH Publishers, New York, N.Y.). Eukaryotic cells are preferred for expression of antibodies and a number of suitable host cell lines have been developed in the art, including Chinese Hamster Ovary (CHO) cells, various COS cell lines, HeLa cells, preferably myeloma cell lines, and transformed B-cells or hybridomas. Expression vectors which express desired sequences in these cells can include expression control sequences, such as an origin of DNA replication, a promoter, an enhancer (Queen et al., 1986, Immunol. Rev., 89, 49-68), and necessary processing sequence sites, such as ribosome binding sites, RNA splice sites, polyadenylation sites, and transcriptional initiation and terminator sequences. Preferred expression control sequences are promoters derived from immunoglobulin genes, Simian Virus (SV) 40, adenovirus, cytomegalovirus, bovine papilloma virus and the like.

The vectors containing the DNA segments of interest can be transferred into the host cell by well-known methods, which vary depending on the type of cellular host. For example, calcium chloride transfection is commonly utilized for prokaryotic cells, whereas calcium phosphate treatment or electroporation may be used for other cellular hosts. (Sambrook et al., 1989, Molecular Cloning, A Laboratory Manual, Cold Spring Harbor, N.Y.).

Once expressed, whole antibodies, dimers derived therefrom, individual light and heavy chains, or other forms of antibodies can be purified according to standard procedures known in the art. Such procedures include, but are not limited to, ammonium sulfate precipitation, the use of affinity columns, routine column chromatography, gel electrophoresis, and the like (see, generally, R. Scopes, “Protein Purification”, Springer-Verlag, N.Y. (1982)). Substantially pure antibodies of at least about 90% to 95% homogeneity are preferred, and antibodies having 98% to 99% or more homogeneity most preferred for pharmaceutical uses. Once purified, the antibodies may then be used therapeutically.

The antibodies of the invention may be used in a therapeutic setting in a pharmaceutical acceptable carrier either alone, or they may be used together with a chemotherapeutic agent such as a non-steroidal anti-inflammatory drug, a corticosteroid, or an immunosuppressant. The antibodies, or complexes derived therefrom, can be prepared in a pharmaceutically accepted dosage form which will vary depending on the mode of administration.

The invention thus embodies a novel composition comprising antibodies that bind with gamma interferon, TNF-alpha or IL-1, alone or in combination, for use in treatment of acne. As stated above, the antibodies can be monoclonal antibodies, polyclonal antibodies, humanized monoclonal antibodies, or monoclonal chimeric antibodies, or a biologically active fragment of any type of antibody herein recited. Generation of each type of antibody is discussed herein and applies to generation of antibodies for use in the novel methods of the invention. Generally, it is preferred that monoclonal humanized antibodies are used because they are non-immunogenic, and thus, will not elicit an immune response. However, any type of antibody may be used in the present invention.

The method of the invention is not intended to be limited to use of antibodies to gamma interferon, TNF-alpha or IL-1. Inhibitors to gamma interferon, inhibitors of TNF alpha and inhibitors of IL-1 are also useful in the method of the invention. Such inhibitors include, but are not limited to, peptides which block the function of gamma interferon, gamma interferon receptor, antibodies to gamma interferon receptors, IFN beta, interleukin-10 (IL-10), removal of IL-6 via an anti-IL-6 antibody (1988, Matsuda et al., Eur. J. Immunol., 18: 951-956) peptides which block the function of TNF-alpha, TNF-alpha receptor, antibodies to TNF-alpha receptor, peptides which block the function of IL-1, receptors for IL-1, antibodies to IL-1 receptors, and any combination thereof. In addition, the present invention encompasses the removal or inhibition of nitric oxide or nitric oxide synthase. Such compounds that could be administered include, but are not limited to free radical scavengers, enzyme inhibitors that inhibit nitric oxide synthase, and an antibody to nitric oxide synthase (1992, Ohsima et al, Biochem. Biophys. Res. Commun. 187: 1291-1297). Nitric oxide inhibitors and nitric oxide synthase inhibitors can also be used to treat other different autoimmune diseases, other than atherosclerosis.

Particularly contemplated additional agents include IFN gamma receptor, TNF alpha receptor, antibodies to IFN gamma receptors, an antibody to a TNF alpha receptor, IFN beta, interleukin-10 (IL-10), and any combination thereof. The isolation of human interferon gamma receptor is well known in the art, and is described in, for example, U.S. Pat. Nos. 5,578,707; 5,221,789; and 4,897,264. Recombinant production of a human interferon gamma receptor, and antibodies that specifically bind a human interferon gamma receptor are well known in the art as well, and is described in, for example, Fountoulakis et al. (1990, J. Biol. Chem. 265: 13268-13275). Also contemplated in the present invention are chimeric interferon gamma receptors, wherein the chimeric interferon gamma receptor comprises a human interferon gamma receptor fused to another protein, such as, but not limited to a human IgG fragment, or the Fc portion of a human immunoglobulin molecule (Fountoulakis et al., 1995, J. Biol. Chem. 270: 3958-3964; Mesa et al., 1995, J. Interferon Cytokine Res. 15: 309-315). Further, the skilled artisan, when equipped with the present disclosure and the methods detailed herein, will readily be able to generate monoclonal, polyclonal and heavy chain antibodies to human interferon gamma receptor, as well as biologically active fragments and the like.

In addition to the administration of an interferon gamma receptor and antibodies that specifically bind an interferon gamma receptor, the present invention encompasses the administration of soluble TNF-alpha receptors, and antibodies thereto. That is, the present invention provides methods for treating acne by administering soluble receptors to TNF-alpha, as well as antibodies to TNF alpha receptors. A soluble TNF-alpha receptor is well known in the art, and isolation from humans is described in, for example, Schall et al. (1990, Cell 61: 361-370). Further, the production of a recombinant soluble TNF-alpha receptor is described in, for example, Gray et al. (1990, Proc. Nat'l. Acad. Sci. USA 87: 7380-7384). The invention further encompasses the administration of antibodies to a TNF-alpha receptor. Such antibodies are well known in the art, and the skilled artisan, when armed with the present invention and the disclosure set forth herein, will readily be able to produce such antibodies. Further, the production of antibodies to a TNF-alpha receptor is described in, for example, Engelmann et al. (J. Biol. Chem. 1990: 265: 14497-14504). Also included in the present invention are a chimeric TNF-alpha receptor, wherein the chimeric protein comprises the 75 kDa or 55 kDa TNF-alpha receptor fused to another protein, such as a human immunoglobulin molecule, or fragments thereof. Such chimeric TNF-alpha receptor fusion proteins are well known in the art, and are described in, for example, Peppel et al. (1991, J. Exp. Med. 174: 1483-1489) and are available commercially, for example, etanercept (Amgen, Inc. Thousand Oaks, Calif.).

IL-10 can be produced and administered according to those methods known in the art, including those set forth in U.S. Pat. Nos. 5,231,012 and 5,328,989.

The present invention further comprises a human IL-1 receptor and antibodies that specifically bind a human IL-1 receptor. Such antibodies and receptors are well known in the art, and are described in, for example, U.S. Pat. No. 4,968,607. Further, the skilled artisan, when equipped with the present disclosure and the methods detailed herein, will readily be able to generate monoclonal, polyclonal and heavy chain antibodies to human IL-1 receptor, as well as biologically active fragments and the like.

The pharmaceutical composition useful for practicing the invention may be administered to deliver a dose of between one microgram per kilogram per day and one hundred milligrams per kilogram per day.

Pharmaceutical compositions that are useful in the methods of the invention may be administered topically or systemically in injectable or other similar formulations. Such injectable formulations include formulations for transdermal, subcutaneous, intramuscular, intravenous, intradermal, cutaneously, and local administration. The present invention further encompasses pharmaceutical compositions for administration via inhalation. In addition to the antibodies to gamma interferon, IL-1 and TNF-alpha, alone or in combination, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer the gamma IFN antibodies according to the methods of the invention.

Compounds comprising antibodies to gamma interferon, TNF-alpha or IL-1, alone or in combination, that can be pharmaceutically formulated and administered to an animal for treatment of autoimmune or inflammatory skin diseases, such as acne, are now described.

The invention encompasses the preparation and use of pharmaceutical compositions comprising antibodies to gamma interferon, antibodies to TNF-alpha or antibodies to IL-1, alone or in combination, as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Controlled- or sustained-release formulations of a pharmaceutical composition of the invention may be made using conventional technology.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein. Ionophoretic administration of the pharmaceutical composition of the invention is considered a form of topical administration herein.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parenterally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for topical administration. Such formulations may, for example, be in the form of liquid, ointment, salve, lotion, cream, and the like, including, for example, a 0.1% to 100% (w/w) solution or suspension of the active ingredient in an aqueous or oily liquid carrier. Such drops may further comprise buffering agents, salts, or one or more other of the additional ingredients described herein. Other administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Preferably, the composition of the invention is administered topically. The composition may be administered as an ointment or in liquid form to the skin on or near the affected area. Preferably, the composition is administered in the form of an application of an antibody to the affected skin. However, the composition comprising antibody to gamma interferon, TNF-alpha or IL-1, alone or in combination, may also be administered parenterally.

As an example, a topical formulation can contain conventional carriers. By way of non-limiting example, the ointments may contain water and one or more hydrophobic carriers selected from, for example, liquid paraffin, polyoxyethylene alkyl ether, propylene glycol and white vaseline. The carrier compositions of the creams are typically based on water and white vaseline, in combination with glycerol and more minor components, e.g. one or more of glycerinemonostearate, PEG-glycerinemonostearate and cetylstearyl alcohol. The gels may by way of example be formulated using isopropyl alcohol and water, suitably in combination with minor components, for example one or more of glycerol and hydroxyethyl cellulose.

In addition, a topical formulation can contain a carrier capable of crossing cellular membranes, such as DMSO in solution with an antibody of the present invention. Such solutions of DMSO can be from about 5% to about 100% DMSO, preferably about 15% to about 90% DMSO, even more preferably about 15% to about 70% DMSO. In addition, DMSO can be used alone, or in combination with an antibody to a cytokine, such as gamma interferon, TNF-alpha and IL-1, a retinoid and an antibiotic.

An ointment, salve, cream, lotion, gel, and the like, can further comprise a moisturizing agent. The moisturizing agent can be a hydrophobic moisturizing agent such as ceramide, borage oil, tocopherol, tocopherol linoleate, dimethicone, glycerine, or a mixture thereof or a hydrophilic moisturizing agent such as hyaluronic acid, sodium peroxylinecarbolic acid, wheat protein, hair keratin amino acids, or a mixture thereof. The pharmaceutical composition can further include a pharmaceutically acceptable carrier or excipient. The pharmaceutical composition can be a gel, paste, cream, lotion, emulsion, or ointment.

Suitable dosage forms for topical administration include, but are not limited to, dispersions, lotions; creams; gels; pastes; powders; aerosol sprays; syrups or ointments on sponges or cotton applicators; and solutions or suspensions in an aqueous liquid, non-aqueous liquid, oil-in-water emulsion, or water-in-oil liquid emulsion. Because of its ease of administration, a cream, lotion, or ointment represents the most advantageous topical dosage unit form, in which case liquid pharmaceutical carriers may be employed in the composition. These creams, lotions, or ointments, may be prepared as rinse-off or leave-on products, as well as two stage treatment products for use with other skin cleansing or managing compositions. The compositions can be administered as a rinse-off product in a higher concentration form, such as a gel, and then a leave-on product in a lower concentration to avoid irritation of the skin. Each of these forms is well understood by those of ordinary skill in the art, such that dosages may be easily prepared to incorporate the pharmaceutical composition of the invention.

The compositions of the invention may be prepared by any of the methods of pharmacy, but all methods include the step of bringing into association the carrier(s) with the active ingredient, which constitutes one or more necessary ingredients. In general, the compositions are prepared by uniformly admixing the active ingredient with liquid carriers or finely divided solid carriers or both, and then, if necessary, shaping the product into the desired presentation.

The present invention further encompasses administration of an antibody via a transdermal patch or other forms of transdermal administration. The methods of the present invention include the transdermal administration of an antibody to gamma interferon, an antibody to TNF-alpha, and an antibody to IL-1, alone or in combination. Suitable formulations for transdermal administration are known in the art, and may be employed in the methods of the present invention. For example, suitable transdermal patch formulations for the administration of a pharmaceutical compound are described in, for example, U.S. Pat. No. 4,460,372 to Campbell et al., U.S. Pat. No. 4,573,996 to Kwiatek et al., U.S. Pat. No. 4,624,665 to Nuwayser, U.S. Pat. No. 4,722,941 to Eckert et al., and U.S. Pat. No. 5,223,261 to Nelson et al.

One suitable type of transdermal patch for use in the methods of the present invention encompasses a suitable transdermal patch includes a backing layer which is non-permeable, a permeable surface layer, an adhesive layer substantially continuously coating the permeable surface layer, and a reservoir located or sandwiched between the backing layer and the permeable surface layer such that the backing layer extends around the sides of the reservoir and is joined to the permeable surface layer at the edges of the permeable surface layer. The reservoir contains an antibody to a cytokine, such as an antibody to gamma interferon, an antibody to TNF-alpha and an antibody to IL-1, alone or in combination, and is in fluid contact with the permeable surface layer. The transdermal patch is adhered to the skin by the adhesive layer on the permeable surface layer, such that the permeable surface layer is in substantially continuous contact with the skin when the transdermal patch is adhered to the skin. While the transdermal patch is adhered to the skin of the subject, the antibodies contained in the reservoir of the transdermal patch is transferred via the permeable surface layer, from the reservoir, through the adhesive layer, and to the skin of the patient. The transdermal patch may optionally also include one or more penetration-enhancing agents in the reservoir that enhance the penetration of the antibodies through the skin.

Examples of suitable materials which may comprise the backing layer are well known in the art of transdermal patch delivery, and any conventional backing layer material may be employed in the transdermal patch of the instant invention. Specific examples of suitable backing layer materials include but are not limited to polyester film, such as high density polyethylene, low density polyethylene or composites of polyethylene; polypropylene; polyvinyl chloride, polyvinylidene chloride; ethylene-vinyl acetate copolymers; and the like.

Examples of suitable permeable surface layer materials are also well known in the art of transdermal patch delivery, and any conventional material which is permeable to the active ingredient to be administered, i.e., antibodies, may be employed in the transdermal patch of the instant invention. Specific examples of suitable materials for the permeable surface layer include but are not limited to dense or microporous polymer films such as those comprised of polycarbonates, polyvinyl chlorides, polyamides, modacrylic copolymers, polysulfones, halogenated polymers, polychloroethers, acetal polymers, acrylic resins, and the like. Specific examples of these types of conventional permeable membrane materials are described in U.S. Pat. No. 3,797,494 to Zaffaroni.

Examples of suitable adhesives which may be coated on the backing layer to provide the adhesive layer are also well known in the art and include, for example pressure sensitive adhesives such as those comprising acrylic and/or methacrylic polymers. Specific examples of suitable adhesives include polymers of esters of acrylic or methacrylic acid (e.g., n-butanol, n-pentanol, isopentanol, 2-methyl butanol, 1-methyl butanol, 1-methyl pentanol, 3-methyl pentanol, 3-methyl pentanol, 3-ethyl butanol, isooctanol, n-decanol, or n-dodecanol esters thereof) alone or copolymerized with ethylenically unsaturated monomers such as acrylic acid, methacrylic acid, acrylamide, methacrylamide, N-alkoxymethyl acrylamides, N-alkoxymethyl methacrylamides, N-t-butylacrylamide, itaconic acid, vinyl acetate, N-branched C.sub.10-24 alkyl maleamic acids, glycol diacrylate, or mixtures of the foregoing; natural or synthetic rubbers such as silicon rubber, styrene-butadiene rubber, butyl-ether rubber, neoprene rubber, nitrile rubber, polyisobutylene, polybutadiene, and polyisoprene; polyurethane elastomers; vinyl polymers such as polyvinyl alcohol, polyvinyl ethers, polyvinyl pyrrolidone, and polyvinyl acetate; ureaformaldehyde resins; phenol formaldehyde resins; resorcinol formaldehyde resins; cellulose derivatives such as ethyl cellulose, methyl cellulose, nitrocellulose, cellulose acetatebutyrate, and carboxymethyl cellulose, and natural gums such as guar, acacia, pectin, starch, destria, gelatin, casein, etc. As will be apparent to those skilled in the art, the adhesive layer should be inert to the active ingredient and should not interfere with the transdermal delivery of an antibody through the permeable surface layer. Pressure sensitive adhesives are preferred for the adhesive layer of the transdermal patch to facilitate the application of the patch to the skin of the subject.

Suitable penetration-enhancing agents are well known in the art as well. Examples of conventional penetration-enhancing agents include alkanols such as ethanol, hexanol, cyclohexanol, and the like; hydrocarbons such as hexane, cyclohexane, isopropylbenzene; aldehydes and ketones such as cyclohexanone, acetamide; N,N-di(lower alkyl)acetamides such as N,N-diethylacetamide, N,N-dimethyl acetamide,; N-(2-hydroxyethyl) acetamide; esters such as N,N-di-lower alkyl sulfoxides; essential oils such as propylene glycol, glycerine, glycerol monolaurate, isopropyl myristate, and ethyl oleate; salicylates; and mixtures of any of the above.

The methods of the invention may further comprise administering one or more additional dermatological agents by a route of administration set forth herein. Any suitable route of administration may be employed for providing the patient with an effective dosage of the disclosed antibodies, but not limited to, oral, intraoral, rectal, parenteral, topical, epicutaneous, transdermal, subcutaneous, intramuscular, intranasal, sublingual, buccal, intradural, intraocular, intrarespiratory, or nasal inhalation and like forms of administration. Preferably, the composition is administered topically.

As an example, an antibody to gamma interferon, an antibody to TNF-alpha and an antibody to IL-1, alone or in combination, can be administered using direct injections to the skin area afflicted by an inflammatory or autoimmune skin disease such as acne vulgaris. Such injections can include cutaneous or subcutaneous injections using a syringe, such as a tuberculin syringe, directly in the affected area. One or more injections can be administered to the area surrounding the affected region.

The compound may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

The antibodies to gamma interferon, TNF-alpha, or IL-1, alone or in combination, may be present in a composition to be administered to the affected skin at a range of concentrations.

A composition comprising an antibody to gamma interferon can be administered to the affected skin several times per day, as disclosed elsewhere herein. Preferably, the composition is administered from one to five times per day, and more preferably, the composition is administered from one to three times per day. Most preferred is administration of the composition three times per day.

Gamma interferon antibodies, TNF alpha antibodies or IL-1 antibodies, alone or in combination, can be administered to the affected skin of a patient for as long as necessary to remedy the effects of the autoimmune of inflammatory reaction. Preferably, the patient receives treatment for about 2 to about 10 days. More preferably, the patient receives treatment for about 4 to about 7 days. The entire treatment of administering gamma interferon, TNF-alpha or IL-1 antibodies, alone or in combination, can be repeated.

The present invention further comprises the administration of additional anti-acne compounds in order to treat acne. An antibody to gamma interferon, TNF-alpha and an antibody to IL-1, alone or in combination can be administered in conjunction with a topical vitamin A derivative, otherwise known as a retinoid, such as adapalene, tazarotene, tretinoin and the like. Topical retinoids are available commercially, and include DIFFERIN (Galderma, Fort Worth Tex.), TAZORAC (Allergan, Irvine, Calif.), RETIN-A MICRO (OrthoNeutrogena, Raritan, N.J.), ACCUTANE (Roche Pharmaceuticals, Nutley, N.J.) and the like.

In addition, the present invention further comprises the administration of and antibiotic against P. acnes. An antibody to gamma interferon, TNF-alpha and an antibody to IL-1, alone or in combination, can be administered in conjunction with a retinoid and an antibiotic, or an antibiotic can be administered with an antibody to gamma interferon, an antibody to TNF-alpha and an antibody to IL-1, wherein the antibody is administered alone or in combination. Preferably, the antibiotic is administered topically, and includes topically administered antibiotics such as erythromycin and clindamycin, which may further be administered with an agent such as benzoyl peroxide. Topical erythromycin is available commercially as AKNE-MYCIN, A/T/S, EMGEL (GlaxoSmithKline, Philadelphia, Pa.), ERYCETTE, ERYDERM, ERYGEL, ERYMAX, ERY-SOL, ERYTHRA-DERM, ETS, STATICIN, THERAMYCIN Z and T-STAT. Topical clindamycin is available commercially as CLEOCIN T GEL, CLEOCIN T LOTION, CLEOCIN T TOPICAL SOLUTION (Pfizer, New York, N.Y.) and CLINDA-DERM.

An antibiotic and a retinoid can be administered in conjunction with an anti-cytokine antibody, or can be administered separately. Preferably, an anti-cyokine antibody is administered according to the dosage and schedule disclosed elsewhere herein and a retinoid and an antibiotic are administered according to the manufacturer's directions.

The present invention further includes kits for the treatment of an autoimmune or inflammatory skin disease, such as acne. The kits of the present invention comprise a compound, including an antibody to gamma interferon, an antibody to TNF-alpha and an antibody to IL-1, an applicator, and instructional materials which describe use of the compound to perform the methods of the invention. Although model kits are described below, the contents of other useful kits will be apparent to the skilled artisan in light of the present disclosure. Each of these kits is contemplated within the present invention. The kits of the present invention can further comprise an antibiotic and a retinoid, either together or separately, along with an anti-cytokine antibody.

In one aspect, the invention includes a kit for treating an inflammatory or autoimmune skin disease, such as acne. The kit is used in the same manner as the methods disclosed herein for the present invention. The kit can be used to administer an antibody to a patient with an inflammatory or autoimmune skin disease, such as acne. The kit comprises an antibody to gamma interferon, an antibody to TNF-alpha and an antibody to IL-1, alone or in combinations disclosed elsewhere herein. As a non-limiting example, the kit can comprise an antibody to gamma interferon. In another non-limiting example, the kit can comprise an antibody to TNF-alpha. In yet another example, the kit can comprise an antibody to IL-1. Other examples of kits contemplated in the present invention comprise an antibody to gamma interferon, and antibody to TNF-alpha, and an antibody to IL-1 in combinations disclosed elsewhere herein. Additionally, the kit comprises an applicator and an instructional material for the use of the kit. These instructions simply embody the examples provided herein.

The kit can further include a pharmaceutically-acceptable carrier. The antibody is provided in an appropriate amount as set forth elsewhere herein. Further, the route of administration and the frequency of administration are as previously set forth elsewhere herein.

As evidenced by the Examples disclosed herein, the present invention is particularly useful in treating a hyperimmune or inflammatory responses resulting from acne. The invention is also useful in preventing an expected autoimmune or inflammatory reaction when the composition of the invention is administered to the patient before an acne vulgaris outbreak. The preferred treatment period is about four days.

As demonstrated by the data disclosed herein, administering gamma interferon antibodies, anti-TNF alpha antibodies or anti-IL-1 antibodies to the affected skin is also effective against pustules and papules caused by an immune reaction to skin bacteria and a hyperproduction of cytokines due to the presence of such bacteria. Hyperproduction of cytokines can also induce an autoimmune response in the skin. Thus, the administration of anti-cytokine antibodies to the skin affected with a disease that causes hyperproduction of cytokines is well within the purview of the present invention.

Definitions

The articles “a” and “an” are used herein to refer to one or to more than one (i.e. to at least one) of the grammatical object of the article. By way of example, “an element” means one element or more than one element.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, N.Y.; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

By the term “applicator” as the term is used herein, is meant any device including, but not limited to, a hypodermic syringe, a pipette, a foam or gauze pad, and the like, for administering an antibody to a human.

By the term “biologically active antibody fragment” is meant a fragment of an antibody which retains the ability to specifically bind to gamma interferon, TNF alpha or IL-1.

The term “interleukin-1” as used herein refers to both interleukin-1-alpha (IL-1α) and interleukin-1-beta (IL-1β), unless specified otherwise.

A “disease” is a state of health of an animal wherein the animal cannot maintain homeostasis, and wherein if the disease is not ameliorated then the animal's health continues to deteriorate. Use of the term disease throughout the application is meant to encompass the terms diseases, disorders, and conditions.

“Treatment” of a disease occurs when the severity of a symptom of the disease, the frequency with which such a symptom is experienced by a patient, or both, is reduced or eliminated. “Treatment” also encompasses prevention of an anticipated disease state. For example, treatment of acne includes use of a composition comprising antibodies to gamma interferon, TNF-alpha or IL-1 after acne formation has already occurred.

By the term “specifically binds,” as used herein, is meant an antibody which recognizes and binds gamma interferon, TNF-alpha or IL-1, but does not substantially recognize or bind other molecules in a sample.

“Autoimmune response” refers to an alteration in the immune system wherein the immune response mounted during a disease state is detrimental to the host. Typically, cells of the immune system or other immune system components such as antibodies produced by the host, recognize “self” antigens as foreign antigens.

A “hyperimmune response” refers to an autoimmune response characterized by an overexpression of one or more cytokines of the immune system.

A pharmaceutical composition is said to be “topically administered” when it is applied directly to the affected area of the skin. Gauze compresses, salves, liquids, lotions, topical sprays and adhesive patches, for example, are applied topically, as are creams and ointments. Ionophoresis is also included as a form of topical administration.

As used herein, an “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

“Recombinant DNA” refers to a polynucleotide having sequences that are not naturally joined together. An amplified or assembled recombinant polynucleotide may be included in a suitable vector, and the vector can be used to transform a suitable host cell. A recombinant DNA polynucleotide may serve a non-coding function (e.g., promoter, origin of replication, ribosome-binding site, etc.) as well.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Anti-IFN-gamma antibodies were produced by immunizing goats with recombinant human IFN-gamma (Peprotech, Rocky Hill, N.J.) using methods well known in the art. Goats were plasmapheresed and the IgG was isolated. F(ab′)₂ fragments were prepared by pepsin digestion and purified by gel filtration. Protein concentration was 33 mg/ml with an IFN-gamma neutralizing activity of >66 μg/ml as determined by a cell growth inhibition assay. F(ab′)₂ fragments were suspended in phosphate buffered saline (PBS).

Patient A. K., 15 years old, presented with multiple infiltrated pustules and papules on the forehead, chin and cheeks characteristic of acne vulgaris. A wet compress comprising gauze soaked in anti-gamma interferon F(ab′)₂ fragments was applied with mild pressure to the afflicted areas for one minute, three times per day for four days. By the second day, a majority of the pustules had desiccated and no new papules appeared. After 4 days of treatment, the infiltrated papules remained, but had paled in color. Patient A. K. noted that during the treatment there was no unpleasant sensation, skin dryness or redness present in other treatments for acne vulgaris.

Patient F. M., 17 years old, presented with multiple small pustules and mild inflammation on the forehead, characteristic of acne vulgaris. A gauze compress comprising anti-gamma interferon F(ab′)₂ fragments was applied with mild pressure to the afflicted areas for one minute, three times per day for four days. After the second day of treatment, the pustules were replaced with thin serous-purulent crusts. After 4 days, the inflammation regressed. On the fifth day, fresh pustules appeared although in a smaller quantity.

The results of the experiments disclosed establish that treatment of inflammatory skin diseases such as acne with antibody to gamma interferon is effective.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating acne in a patient, the method comprising administering to the patient an effective amount of an antibody to gamma interferon, an antibody to tumor necrosis factor alpha, and an antibody to interleukin-1.

2. The method of claim 1, wherein the antibody is selected from the group consisting of a polyclonal antibody, a monoclonal antibody, a humanized antibody, a synthetic antibody, a heavy chain antibody, a biologically active fragment of an antibody, wherein the biologically active fragment is a Fab fragment, a F(ab′)₂ fragment, a Fv fragment, and combinations thereof.

3. The method of claim 1, wherein the antibody is administered by the route selected from the group consisting of intramuscularly, intravenously, intradermally, cutaneously, ionophoretically, topically, locally, and inhalation.

4. The method of claim 3, wherein the antibody is administered topically.

5. The method of claim 4, wherein the antibody is selected from the group consisting of a polyclonal antibody, a monoclonal antibody, a synthetic antibody, a heavy chain antibody, a humanized antibody, a biologically active fragment of an antibody, wherein the biologically active fragment is a Fab fragment, a F(ab′)₂ fragment, a Fv fragment, and combinations thereof.

6. The method of claim 5, wherein the heavy chain antibody is selected from the group consisting of a camelid antibody, a heavy chain disease antibody, and a variable heavy chain immunoglobulin.

7. The method of claim 1, wherein the method further comprises administering an antibiotic.

8. The method of claim 1, wherein the method further comprises administering a retinoid.

9. A method of treating acne in a patient, the method comprising administering to the patient an effective amount of an antibody to tumor necrosis factor alpha, and an antibody to interleukin-1.

10. The method of claim 9, wherein the antibody is selected from the group consisting of a polyclonal antibody, a monoclonal antibody, a humanized antibody, a synthetic antibody, a heavy chain antibody, a biologically active fragment of an antibody, wherein the biologically active fragment is a Fab fragment, a F(ab′)₂ fragment, a Fv fragment, and combinations thereof.

11. The method of claim 9, wherein the antibody is administered by the route selected from the group consisting of intramuscularly, intravenously, intradermally, cutaneously, ionophoretically, topically, locally, and inhalation.

12. The method of claim 11, wherein the antibody is administered topically.

13. The method of claim 12, wherein the antibody is selected from the group consisting of a polyclonal antibody, a monoclonal antibody, a synthetic antibody, a heavy chain antibody, a humanized antibody, a biologically active fragment of an antibody, wherein the biologically active fragment is a Fab fragment, a F(ab′)₂ fragment, a Fv fragment, and combinations thereof.

14. The method of claim 13, wherein the heavy chain antibody is selected from the group consisting of a camelid antibody, a heavy chain disease antibody, and a variable heavy chain immunoglobulin.

15. The method of claim 9, wherein the method further comprises administering an antibiotic.

16. The method of claim 9, wherein the method further comprises administering a retinoid.

17. A method of treating acne in a patient, the method comprising administering to the patient an effective amount of an antibody to gamma interferon and an antibody to interleukin-1.

18. The method of claim 17, wherein the antibody is selected from the group consisting of a polyclonal antibody, a monoclonal antibody, a humanized antibody, a synthetic antibody, a heavy chain antibody, a biologically active fragment of an antibody, wherein the biologically active fragment is a Fab fragment, a F(ab′)₂ fragment, a Fv fragment, and combinations thereof.

19. The method of claim 17, wherein the antibody is administered by the route selected from the group consisting of intramuscularly, intravenously, intradermally, cutaneously, ionophoretically, topically, locally, and inhalation.

20. The method of claim 19, wherein the antibody is administered topically.

21. The method of claim 20, wherein the antibody is selected from the group consisting of a polyclonal antibody, a monoclonal antibody, a synthetic antibody, a heavy chain antibody, a humanized antibody, a biologically active fragment of an antibody, wherein the biologically active fragment is a Fab fragment, a F(ab′)₂ fragment, a Fv fragment, and combinations thereof.

22. The method of claim 21, wherein the heavy chain antibody is selected from the group consisting of a camelid antibody, a heavy chain disease antibody, and a variable heavy chain immunoglobulin.

23. The method of claim 17, wherein the method further comprises administering an antibiotic.

24. The method of claim 17, wherein the method further comprises administering a retinoid.

25. A method of treating acne in a patient, the method comprising administering to the patient an effective amount of an antibody to gamma interferon and an antibody to tumor necrosis factor alpha.

26. The method of claim 25, wherein the antibody is selected from the group consisting of a polyclonal antibody, a monoclonal antibody, a humanized antibody, a synthetic antibody, a heavy chain antibody, a biologically active fragment of an antibody, wherein the biologically active fragment is a Fab fragment, a F(ab′)₂ fragment, a Fv fragment, and combinations thereof.

27. The method of claim 25, wherein the antibody is administered by the route selected from the group consisting of intramuscularly, intravenously, intradermally, cutaneously, ionophoretically, topically, locally, and inhalation.

28. The method of claim 27, wherein the antibody is administered topically.

29. The method of claim 28, wherein the antibody is selected from the group consisting of a polyclonal antibody, a monoclonal antibody, a synthetic antibody, a heavy chain antibody, a humanized antibody, a biologically active fragment of an antibody, wherein the biologically active fragment is a Fab fragment, a F(ab′)₂ fragment, a Fv fragment, and combinations thereof.

30. The method of claim 29, wherein the heavy chain antibody is selected from the group consisting of a camelid antibody, a heavy chain disease antibody, and a variable heavy chain immunoglobulin.

31. The method of claim 25, wherein the method further comprises administering an antibiotic.

32. The method of claim 25, wherein the method further comprises administering a retinoid.

33. A method of treating acne in a patient, the method comprising administering to the patient an effective amount of an antibody to gamma interferon.

34. The method of claim 33, wherein the antibody is selected from the group consisting of a polyclonal antibody, a monoclonal antibody, a humanized antibody, a synthetic antibody, a heavy chain antibody, a biologically active fragment of an antibody, wherein the biologically active fragment is a Fab fragment, a F(ab′)₂ fragment, a Fv fragment, and combinations thereof.

35. The method of claim 34, wherein the antibody is administered by the route selected from the group consisting of intramuscularly, intravenously, intradermally, cutaneously, ionophoretically, topically, locally, and inhalation.

36. The method of claim 35, wherein the antibody is administered topically.

37. The method of claim 36, wherein the antibody is selected from the group consisting of a polyclonal antibody, a monoclonal antibody, a synthetic antibody, a heavy chain antibody, a humanized antibody, a biologically active fragment of an antibody, wherein the biologically active fragment is a Fab fragment, a F(ab′)₂ fragment, a Fv fragment, and combinations thereof.

38. The method of claim 37, wherein the heavy chain antibody is selected from the group consisting of a camelid antibody, a heavy chain disease antibody, and a variable heavy chain immunoglobulin.

39. The method of claim 33, wherein the method further comprises administering an antibiotic.

40. The method of claim 33, wherein the method further comprises administering a retinoid.

41. A method of treating acne in a patient, the method comprising administering to the patient an effective amount of an antibody to interleukin-1.

42. The method of claim 41, wherein the antibody is selected from the group consisting of a polyclonal antibody, a monoclonal antibody, a humanized antibody, a synthetic antibody, a heavy chain antibody, a biologically active fragment of an antibody, wherein the biologically active fragment is a Fab fragment, a F(ab′)₂ fragment, a Fv fragment, and combinations thereof.

43. The method of claim 41, wherein the antibody is administered by the route selected from the group consisting of intramuscularly, intravenously, intradermally, cutaneously, ionophoretically, topically, locally, and inhalation.

44. The method of claim 43, wherein the antibody is administered topically.

45. The method of claim 44, wherein the antibody is selected from the group consisting of a polyclonal antibody, a monoclonal antibody, a synthetic antibody, a heavy chain antibody, a humanized antibody, a biologically active fragment of an antibody, wherein the biologically active fragment is a Fab fragment, a F(ab′)₂ fragment, a Fv fragment, and combinations thereof.

46. The method of claim 45, wherein the heavy chain antibody is selected from the group consisting of a camelid antibody, a heavy chain disease antibody, and a variable heavy chain immunoglobulin.

47. The method of claim 41, wherein the method further comprises administering an antibiotic.

48. The method of claim 41, wherein the method further comprises administering a retinoid.

49. A method of treating acne in a patient, the method comprising administering to the patient an effective amount of an antibody to tumor necrosis factor alpha.

50. The method of claim 49, wherein the antibody is selected from the group consisting of a polyclonal antibody, a monoclonal antibody, a humanized antibody, a synthetic antibody, a heavy chain antibody, a biologically active fragment of an antibody, wherein the biologically active fragment is a Fab fragment, a F(ab′)₂ fragment, a Fv fragment, and combinations thereof.

51. The method of claim 50, wherein the antibody is administered by the route selected from the group consisting of intramuscularly, intravenously, intradermally, cutaneously, ionophoretically, topically, locally, and inhalation.

52. The method of claim 51, wherein the antibody is administered topically.

53. The method of claim 52, wherein the antibody is selected from the group consisting of a polyclonal antibody, a monoclonal antibody, a synthetic antibody, a heavy chain antibody, a humanized antibody, a biologically active fragment of an antibody, wherein the biologically active fragment is a Fab fragment, a F(ab′)₂ fragment, a Fv fragment, and combinations thereof.

54. The method of claim 53, wherein the heavy chain antibody is selected from the group consisting of a camelid antibody, a heavy chain disease antibody, and a variable heavy chain immunoglobulin.

55. A kit for treating acne in a patient, said kit comprising an antibody to gamma interferon, an antibody to tumor necrosis factor alpha, an antibody to interleukin-1, and a pharmaceutically acceptable carrier, said kit further comprising an applicator, and an instructional material for the use thereof.

56. A kit for treating acne in a patient, said kit comprising an antibody to tumor necrosis factor alpha, an antibody to interleukin-1, and a pharmaceutically acceptable carrier, said kit further comprising an applicator, and an instructional material for the use thereof.

57. A kit for treating acne in a patient, said kit comprising an antibody to gamma interferon, an antibody to interleukin-1, and a pharmaceutically acceptable carrier, said kit further comprising an applicator, and an instructional material for the use thereof.

58. A kit for treating acne in a patient, said kit comprising an antibody to gamma interferon, an antibody to tumor necrosis factor alpha, and a pharmaceutically acceptable carrier, said kit further comprising an applicator, and an instructional material for the use thereof.

59. A kit for treating acne in a patient, said kit comprising an antibody to gamma interferon and a pharmaceutically acceptable carrier, said kit further comprising an applicator, and an instructional material for the use thereof.

60. A kit for treating acne in a patient, said kit comprising an antibody to interleukin-1 and a pharmaceutically acceptable carrier, said kit further comprising an applicator, and an instructional material for the use thereof.

61. A kit for treating acne in a patient, said kit comprising an antibody to tumor necrosis factor alpha, and a pharmaceutically acceptable carrier, said kit further comprising an applicator, and an instructional material for the use thereof.
